(19) United States
(12) Patent Application Publication
(10) Pub. No.: US 2011/0105828 A1
(43) Pub. Date: May 5, 2011

(54) Methods of treating a body part

(75) Inventors: Robert Perless, Greenwich, CT (US); Halina Snowball, Greenwich, CT (US)

(73) Assignee: Magnetic Acupuncture, LLC, Greenwich, CT (US)

(21) Appl. No.: 12/589,943

(22) Filed: Oct. 30, 2009

(51) Int. Cl. A61N 2/08

(52) U.S. Cl. 600/15

(57) ABSTRACT

The invention generally relates to devices and methods for treating a body part using magnets. In certain embodiments, the invention provides a device for treating a body part including a carrier and a plurality of magnets, in which the magnets are configured to correspond with a plurality of acupuncture meridian points upon application of the device to the body part.

### FIELD OF THE INVENTION

The invention generally relates to devices and methods for treating a body part using magnets.

### BACKGROUND

The medical profession relies principally on ingestion of pain-killing or anti-inflammatory drugs for treatment of injured body parts, e.g., swelling, inflammation, pain, aches, soreness, or stiffness of body parts. Topical applications of analgesic creams or lotions and/or heat are sometimes recommended, and some patients receive acupuncture therapy.

Acupuncture therapy involves placement of needles to redirect and improve flow of energy in a person's body. The needles must be inserted into the skin of the person at selected points by highly skilled professionals. Since the needles penetrate the skin, there is a degree of discomfort for a person receiving therapy. Additionally, the acupuncturist must guard against the risk of infection to himself and to the person receiving therapy. Further, the needles must be removed before the person leaves a treatment office.

There is an unmet need for devices and methods for treating a body part.

### SUMMARY

The invention generally relates to devices and methods for treating a body part using magnets, thereby providing noninvasive devices and methods for treating body parts, particularly injured body parts. A person's body produces small magnetic charges resulting from movement of ions throughout the body, and a precise electromagnetic balance is maintained by the body for all functions and processes. Swelling, inflammation, and pain are the result of an imbalance in the electromagnetic functions of the body. The devices and methods of the invention use magnets configured to correspond with a plurality of acupuncture meridian points to deliver magnetic energy to body parts. The configuration of the magnets stimulates multiple points across the meridians resulting in magnetic energy (magnetic flux) penetrating the body part to energize, speed circulation of, and accelerate oxygenation of blood, thereby generating moderate heat, increasing blood flow, and relaxing muscles and capillaries. These actions result in reduction of pain and speed healing of the injured body part. Devices and methods of the invention can also be used as a preventative by applying devices of the invention to body parts that are stressed during physical activity, such as knees, elbows, soft tissue (e.g., tendons), and ankles, prior to occurrence of an injury the body part.

An aspect of the invention provides a device for treating a body part including a carrier and a plurality of magnets, in which the magnets are configured to correspond with a plurality of acupuncture meridian points upon application of the device to the body part. Another aspect of the invention provides a device for treating a body part including a carrier and a magnet, in which the magnet is configured to correspond with a plurality of acupuncture meridian points upon application of the device to the body part. The device can be used as a preventative and be applied to non-injured body parts. Alternatively the device can be applied to an injured body part.

The magnetic can be any type of magnet. One of skill in the art can determine the appropriate magnets to use based upon the attributes of the person receiving treatment (e.g., sex, age, weight, or height) and/or the body part to be treated (e.g., knee, shoulder, elbow, or ankle). Exemplary magnets are rare earth magnets, i.e., magnets made from alloys of rare earth elements such as scandium and yttrium. The magnets can be magnetically oriented in the same direction about the body part. Alternatively, the magnets can be oriented in different magnetic directions about the body part.

The magnets can be arranged about the carrier in numerous configurations. The magnets can be affixed to an exterior surface or an interior surface of the carrier. In certain embodiments, one or more magnets contact the subject's skin. To protect a person's skin from irritation or allergic reaction, and to protect each magnet from corrosion, at least a portion of a magnet is covered with an inert metal. The magnets can also be contained within the carrier, such that no surface of the magnet directly contacts a person's skin.

The magnets can be of any shape or any size. One of skill in the art can determine the appropriate shape or size of the magnets to be used depending on the body part to be treated. In certain embodiments, at least one magnet includes a planar surface, and the planar surface of the magnet is applied to the body part. In other embodiments, at least one magnet includes a pointed surface, and the pointed surface of the magnet is applied to the body part.

The carrier can be composed of any material. For example, the carrier can be composed of an elastic material, such as a polymeric material. Exemplary carriers include a bandage, a brace, a wrap, or a patch.

Another aspect of the invention provides a method for treating a body part of a subject including applying a plurality of magnets to a body part of a subject, in which the magnets are configured to correspond to a plurality of meridians, thereby to stimulate multiple points across the body part and trigger a response. The method can further include performing acupuncture on the body part. The method can also further include performing acupuncture or acupressure on an area of the subject that is different from the body part, in which the area is associated with the body part through one or more meridians.

Another aspect of the invention provides a device for treating a body part including a carrier and a plurality of magnets, in which the magnets are configured to result in periosteal stimulation of the body part upon application of the device to the body part.

### BRIEF DESCRIPTION OF THE DRAWINGS

FIG. 1 is a drawing showing meridians in a human body and certain acupuncture points along these meridians.

FIGS. 2A and 2B are drawings showing the urinary bladder meridian and acupuncture points along this meridian.

FIG. 3 is a drawing showing the gall bladder meridian and acupuncture points along this meridian.

FIGS. 4A and 4B are drawings showing the stomach meridian and acupuncture points along this meridian.

FIG. 5 is a drawing showing the large intestine meridian and acupuncture points along this meridian.

FIG. 6 is a drawing showing the triple heater meridian and acupuncture points along this meridian.

FIG. 7 is a drawing showing the small intestine meridian and acupuncture points along this meridian.

FIGS. 8A and 8B are drawings showing the kidney meridian and acupuncture points along this meridian.

FIG. 9 is a drawing showing the liver meridian and acupuncture points along this meridian.

FIGS. 10A and 10B are drawings showing the spleen meridian and acupuncture points along this meridian.

FIG. 11 is a drawing showing the lung meridian and acupuncture points along this meridian.

FIG. 12 is a drawing showing the pericardium meridian and acupuncture points along this meridian.

FIG. 13 is a drawing showing the heart meridian and acupuncture points along this meridian.

FIG. 14 is a drawing showing the governing vessel meridian and acupuncture points along this meridian.

FIG. 15 is a drawing showing the conception vessel meridian and acupuncture points along this meridian.

FIG. 16 panels A-C is a set of photographs showing multiple acupuncture points along the bladder principle meridian that will be stimulated by an upper thoracic brace in order to provide treatment to the muscles of the thoracic cavity of a patient. Panel A shows the acupuncture points along the principle meridian. Panels B and C provide different views of a patient wearing the upper thoracic brace.

FIG. 17 panels A-C is a set of photographs showing multiple acupuncture points along the bladder principle meridian that will be stimulated by a lumbar brace in order to provide treatment to the lumbar muscles of a patient. Panel A shows the acupuncture points along the principle meridian. Panels B and C provide different views of a patient wearing the lumbar brace.

FIG. 18 panels A-C is a set of photographs showing multiple acupuncture points along the Heart meridian, Master Heart meridian, Triple Heater meridian, and Large Intestine meridian that will be stimulated by an elbow brace in order to provide treatment to the elbow of a patient. Panel A shows the acupuncture points along the Heart meridian and Master Heart meridians. Panel B shows the acupuncture point along the Triple Heater meridian. Panel C shows the acupuncture points along the Large Intestine meridian. Panels D and E provide different views of a patient wearing the elbow brace.

FIG. 19 panels A-C is a set of photographs showing a subject wearing a knee brace containing magnets that are configured to stimulate acupuncture points across multiple meridians for treating a knee joint. These photographs show the specific configuration of the magnets about the brace. Panel A is a front view, while panels B and C are side views.

### DETAILED DESCRIPTION

The invention generally relates to devices and methods for treating a body part using magnets. The body part can be any anatomical structure of a human body. A body part includes organs, muscles, bones, or soft/connective tissue (e.g., cartilage, joints, ligaments, or tendons). Body parts to be treated are known to one of skill in the art and can be found in any anatomy textbook, such as Gray's Anatomy: The Anatomical Basis of Clinical Practice, 40th edition (2008) or Woodburne et al. Essentials of Human Anatomy, 9th edition, Oxford University Press (1994), the contents of each of these is incorporated by reference herein in their entirety. Exemplary body parts to be treated include neck, shoulder, arm, elbow, wrist, hand, back, chest, abdomen, hip, buttock, leg, thigh, knee, calf, heel, ankle, or foot.

An aspect of the invention provides a device for treating the body part including a carrier and a plurality of magnets. The device can be used as a preventative and be applied to non-injured body parts. Alternatively the device can be applied to an injured body part.

The carrier can be composed of any plastic or stretchable or elastic material. An exemplary material is a stretchable fabric, such as nylon, LYCRA (spandex), polymer based materials (e.g., polyester), or rubber (e.g., latex rubber). These materials can be blended with other fabrics (e.g., cotton, poplin, twill, linen, or denim) to produce a blended stretchable material, such as stretch cotton fabric, stretch poplin fabric, stretch twill fabric, stretch denim fabric, stretch linen fabric, or any other stretch woven fabric. Alternatively, the carrier can be composed of a non-pliable material (e.g, cotton, linen, or denim) and held to the body part by at least one strap, clip, or clasp. Exemplary carriers include bandages, braces, wraps, or patches, each of which is commercially available from any drug store such as Walgreens, Target, or Wal-Mart.

In certain embodiments, the carrier is a tubular shape and is held to the body part by the compressive force of the carrier material. In other embodiments, the carrier member includes fastening members to hold the device to the body part. The fastening members can be any hardware device that mechanically joins or affixes two ends of the carrier together, such as hook/loop fasteners, clips, clasps, straps, VELCRO, ties, etc. In a particular embodiment, the carrier is a tubular shape and includes VELCRO straps for adjustment of the fit of the device to the body part. In certain embodiments, the carrier includes an adhesive and is held to the body part by the adhesive, such as with a patch.

The carrier is shaped to conform to the body part being treated. For example, the carrier can have a tubular shape to slide over a knee, elbow, arm, or leg. Alternatively, the carrier can have a planar shape to cover a shoulder. In certain embodiments, the carrier contains portions having different shapes, for example a tubular portion and a planar portion, the device being configured such that the carrier would cover a shoulder and wrap around an upper part of an arm. In certain embodiments, the carrier is custom fitted for the body part to be treated and the person to be treated.

The carrier holds a plurality of magnets. The magnets can be affixed to an exterior surface of the carrier or an interior surface of the carrier. Alternatively, the magnets can be placed within the carrier such that the magnets are enveloped by the carrier and the magnets do not directly contact a person's skin. The magnets can be affixed to the carrier using any method known in the art. For example, the magnets can be adhered to the carrier using commercially available adhesives, such as glues, pastes, and epoxies. In a particular embodiment, a punch is used to insert snaps into certain positions in the carrier and the magnets are configured to snap fit into the snaps. In other embodiments, the magnets are stitched into the carrier.

The carrier can be configured such that all the magnets are affixed to the exterior surface of the carrier. The carrier can be configured such that all the magnets are affixed to the interior surface of the carrier and contact a person's skin. The carrier can be configured such that all the magnets are placed within the carrier. The carrier can be configured to include a combination of any of the above, e.g., some magnets affixed to the exterior surface of the carrier, some magnets affixed to the interior surface of the carrier, and some magnets placed within the carrier. To protect a person's skin from irritation or allergic reaction, and to protect each magnet from corrosion, at least a portion of a magnet is covered with an inert metal, e.g., a gold plating.

The magnets to be used in the devices of the invention can be any magnets that can stimulate a biological response in the body part to which the device is applied. Exemplary magnets are permanent magnets. Permanent Magnets are classified into four types based on composition: Neodymium Iron Boron (NdFeB or NIB); Samarium Cobalt (SmCo); Alnico; and Ceramic or Ferrite. NIB and SmCo (commercially available from Stanford Magnets, Aliso Viejo, Calif.) are the strongest types of magnets and are examples of a class of magnets also known as rare earth magnets. Rare earth magnets are strong permanent magnets made from alloys of rare earth elements. Rare earth elements or rare earth metals include seventeen chemical elements: scandium, yttrium, and the fifteen lanthanoids. Rare earth magnets are commercially available from Stanford Magnets (Aliso Viejo, Calif.). In certain embodiments, the device of the invention includes at least one rare earth magnet. In certain embodiments, the device includes only rare earth magnets.

Alnico is a compound made of ALuminium, NIckel and CObalt. Alnico magnets (commercially available from Stanford Magnets, Aliso Viejo, Calif.) are commonly used magnets that are not as strong as NIB and SmCo magnets, however, Alnico magnets are least affected by temperature.

Permanent magnets can also be classified into injection molded and flexible magnets. Injection molded magnets are a composite of various types of resin and magnetic powders, allowing parts of complex shapes to be manufactured by injection molding. The physical and magnetic properties of the product depend on the raw materials, but are generally lower in magnetic strength and resemble plastics in their physical properties. Flexible magnets are similar to injection molded magnets, using a flexible resin or binder such as vinyl, and produced in flat strips or sheets. These magnets are lower in magnetic strength but can be very flexible, depending on the binder used. Flexible magnets are useful for treating a curved body part in which the magnet is configured in the carrier to conform to the curve of the body part, such as a shoulder.

In certain embodiments, the magnet is a temporary magnet. Temporary magnets are materials that act like permanent magnets in the presence of a strong magnetic field. Magnetism of these materials is lost when the magnetic field is removed, i.e., temporary magnets.

In certain embodiments, the magnet is an electromagnet and the device includes a coil of wire that is capable of carrying an electric current. The electricity in the current produces a magnetic field. The strength of the magnet is directly proportional to the strength of the current and the number of coils of wire. The polarity of the magnet depends on the direction of flow of current. While the current flows, the core behaves like a magnet. However, as soon as the current stops, the core is demagnetized. Embodiments of the device that include electromagnets allow for pulsed stimulation of the body part.

In certain embodiments, the device includes only a single type of magnet, such as only permanent magnets, or only rare earth magnets. In other embodiments, the device includes more than one type of magnet, such as permanent magnets, temporary magnets, and/or electromagnets. In other embodiments, the device is a combination of permanent magnets, such as rare earth magnets, and electromagnets. In such an embodiment, the rare earth magnets provide continuous stimulation of certain acupuncture points while the electromagnets provide pulsed or continuous stimulation of certain acupuncture points depending on whether the electromagnets are receiving a current.

The magnets can be made into any shape. The device can include magnets all of the same shape, or the device can include magnets of different shapes. Exemplary shapes include round bars, rectangles, horseshoes, donuts, rings, disks and other custom shapes. One of skill in the art can determine the appropriate shape or size of the magnets to be used depending on the person receiving treatment (e.g., sex, body type, weight, or height) and/or the body part to be treated (e.g., knee, shoulder, elbow, or ankle). In certain embodiments, at least one magnet includes a planar surface, and the planar surface of the magnet is applied to the body part. In other embodiments, at least one magnet includes a pointed surface, and the pointed surface of the magnet is applied to the body part.

The magnets can have numerous orientations about the carrier. One of skill in the art will be able to determine the orientation of the magnets about the carrier depending on the body part to be treated and the treatment to be administered. For example, the magnets can be magnetically oriented in the same direction about the body part. Alternatively, the magnets can be oriented in different magnetic directions about the body part. For most treatments, the north pole of the magnet normally is directed toward the area being treated, but reversing or even alternating the polarity of the magnet may be used in some embodiments for possible improved treatment results. The magnets can include a mark, such as a dimple or a recess on the surface of the magnet, to identify the north or south pole of the magnet.

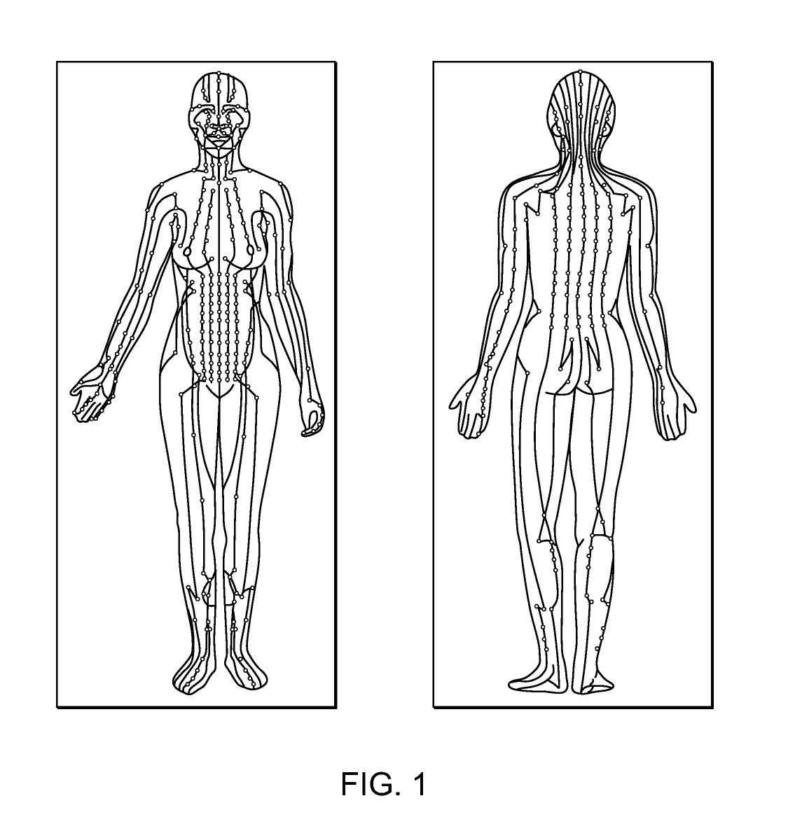

The magnets are configured about the carrier to correspond with a plurality of acupuncture meridian points upon application of the device to the body part. A human body includes meridians that are channels/conduits through which chi (Chinese term for the body's energy) circulates throughout the body. There are twelve main meridians, six of which are yin and six are yang and numerous minor ones, which form a network of energy channels throughout the body. Each meridian is related to, and named after, an organ or function, the main meridians are: lung, kidney, stomach, spleen, heart, small intestine, large intestine, gall bladder, urinary bladder, san jiao (three heater) and pericardium (heart protector/ or circulation sex meridian). There are also eight extraordinary channels that are considered to be reservoirs supplying chi and blood to the twelve regular channels. The eight extraordinary channels are believed to have a strong connection to the kidney. Meridians in a human body are shown in FIG. 1.

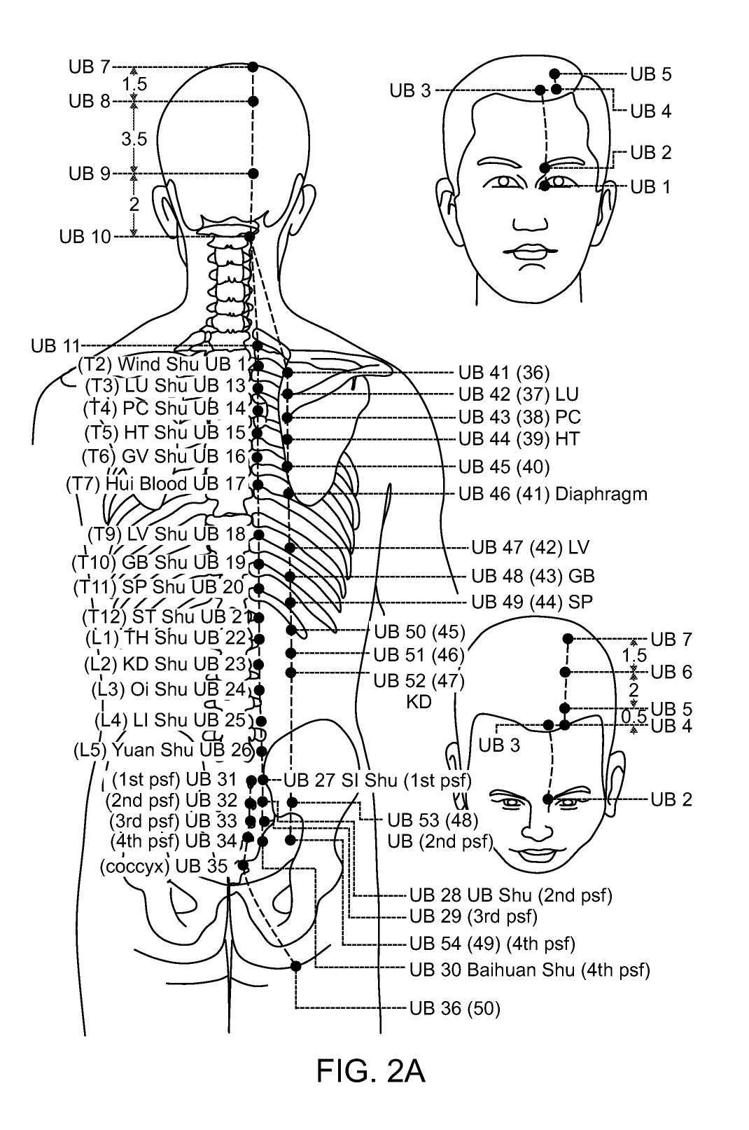

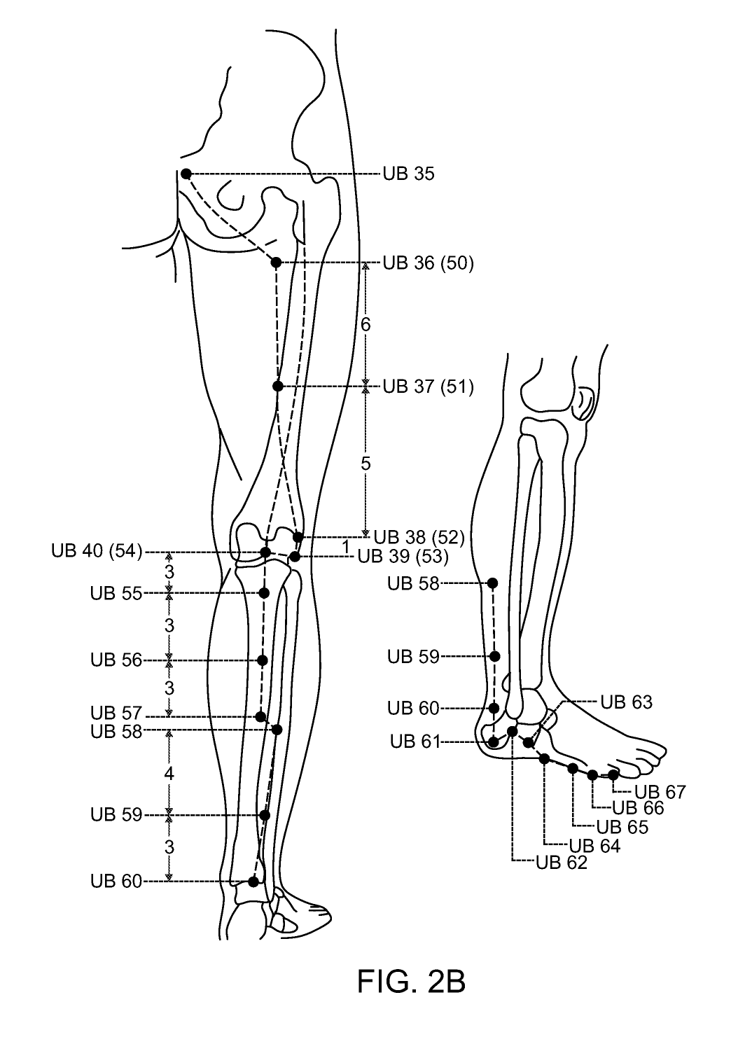

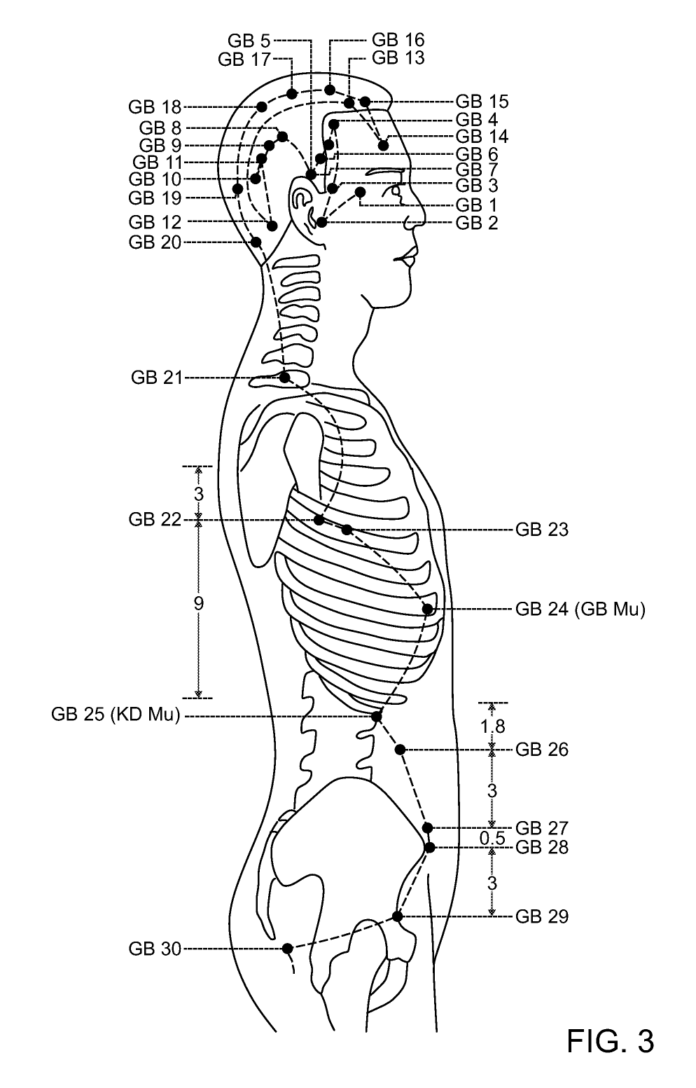

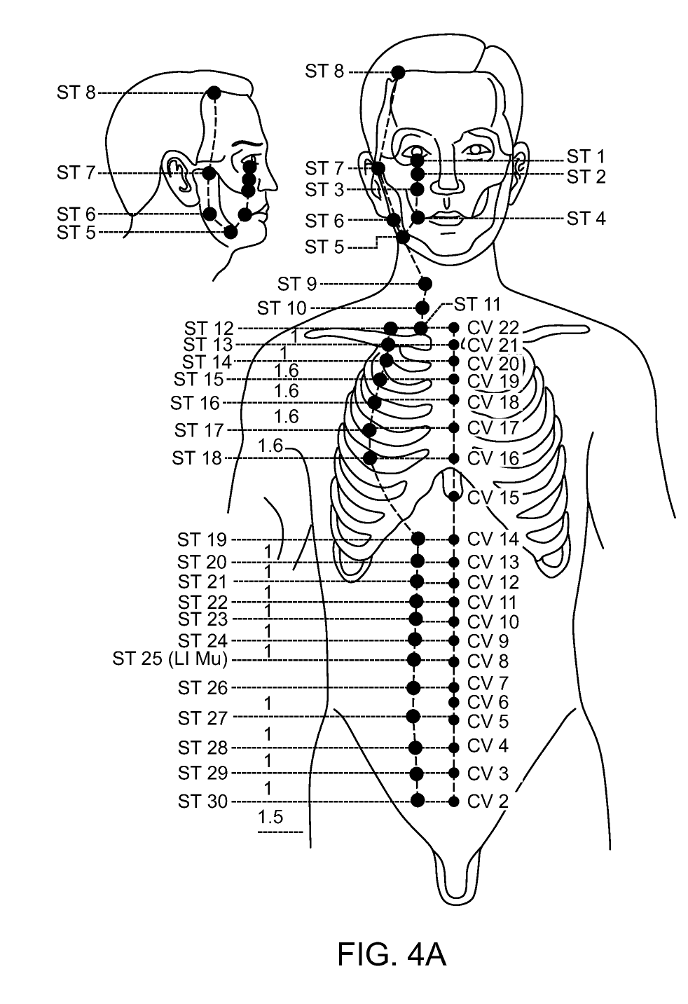

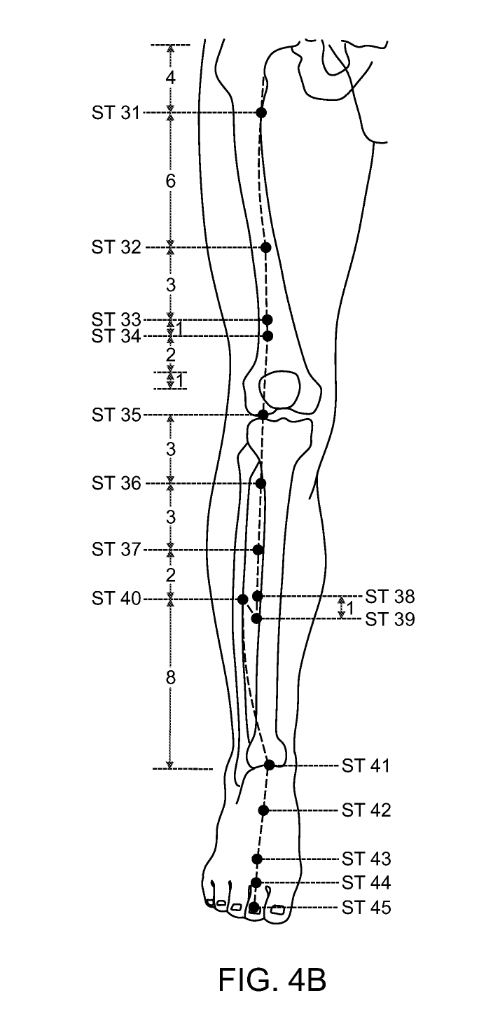

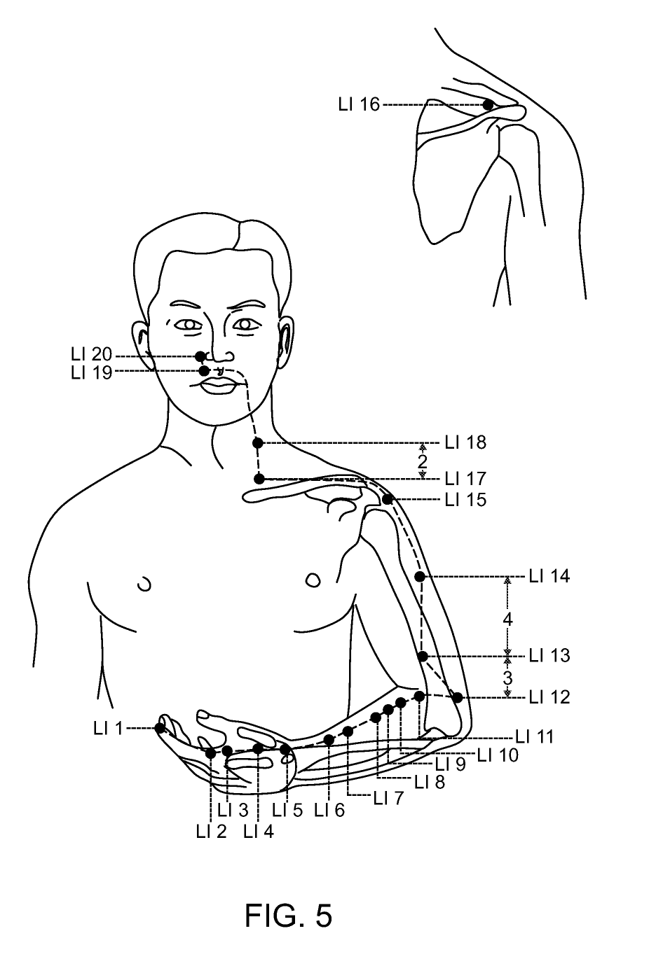

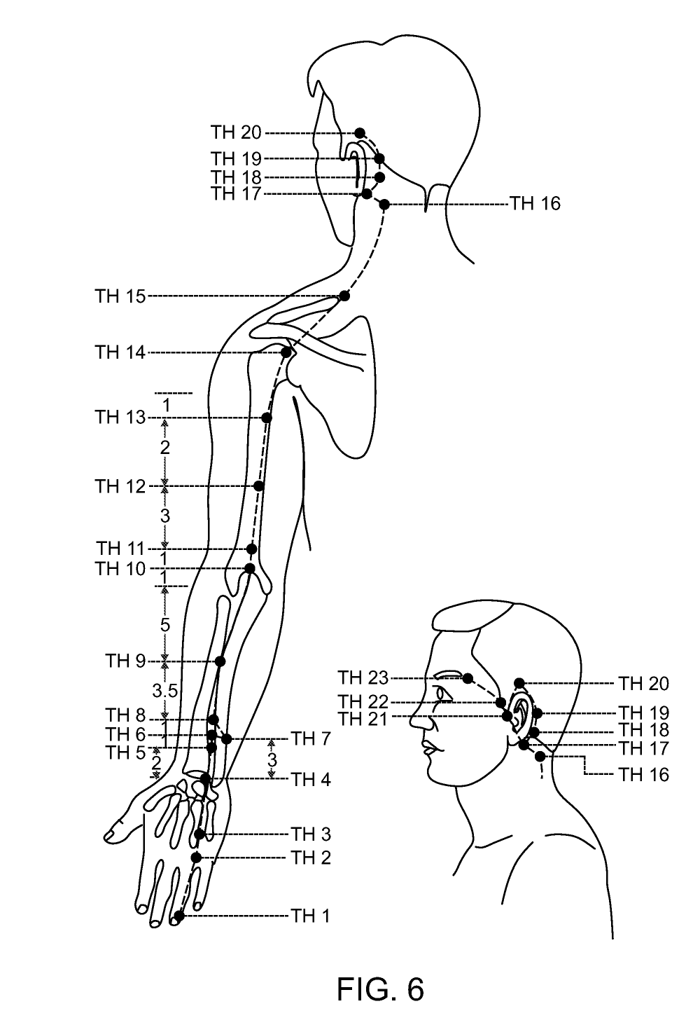

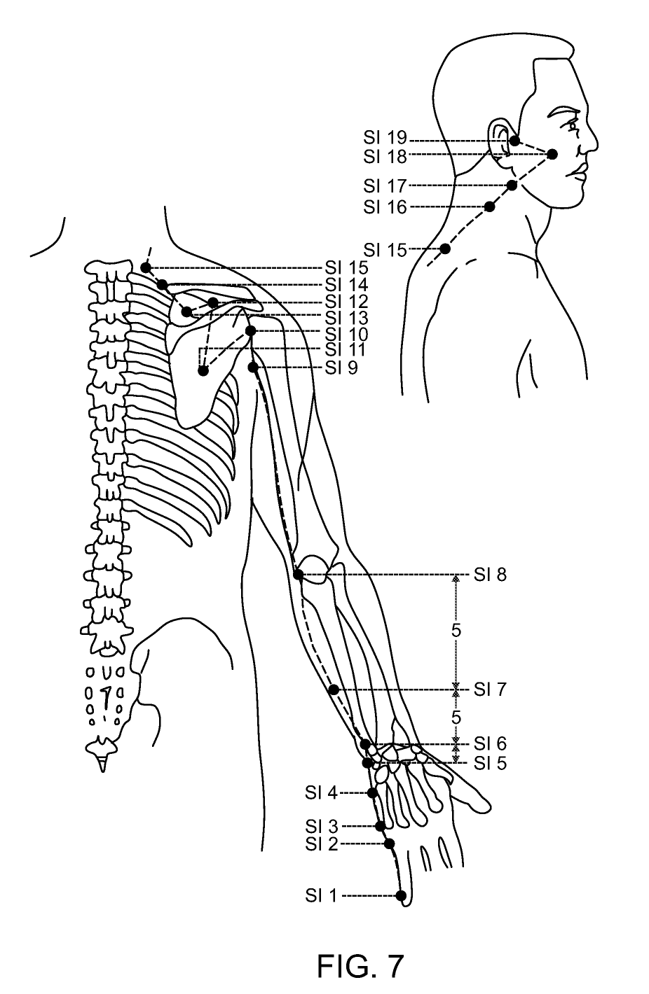

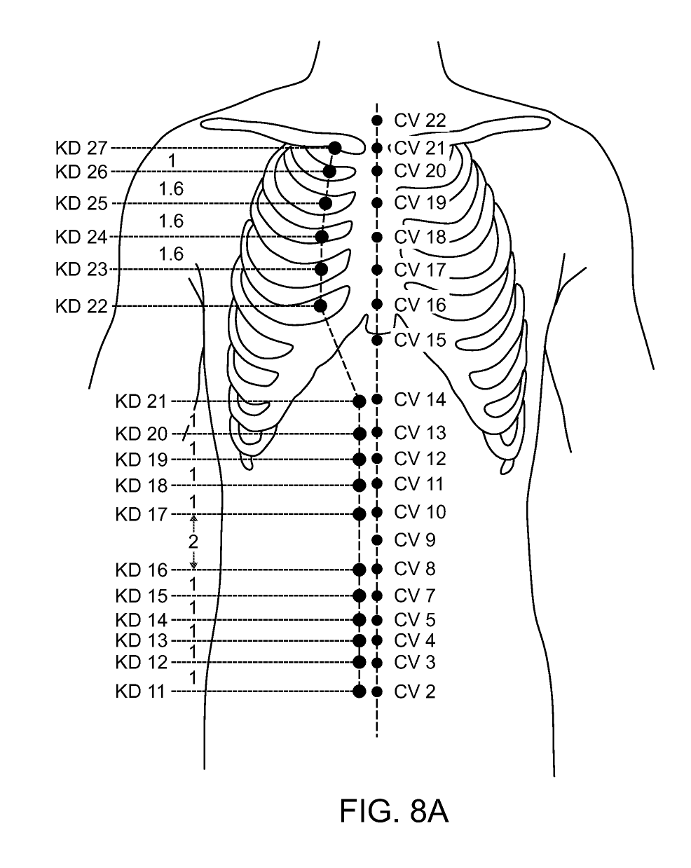

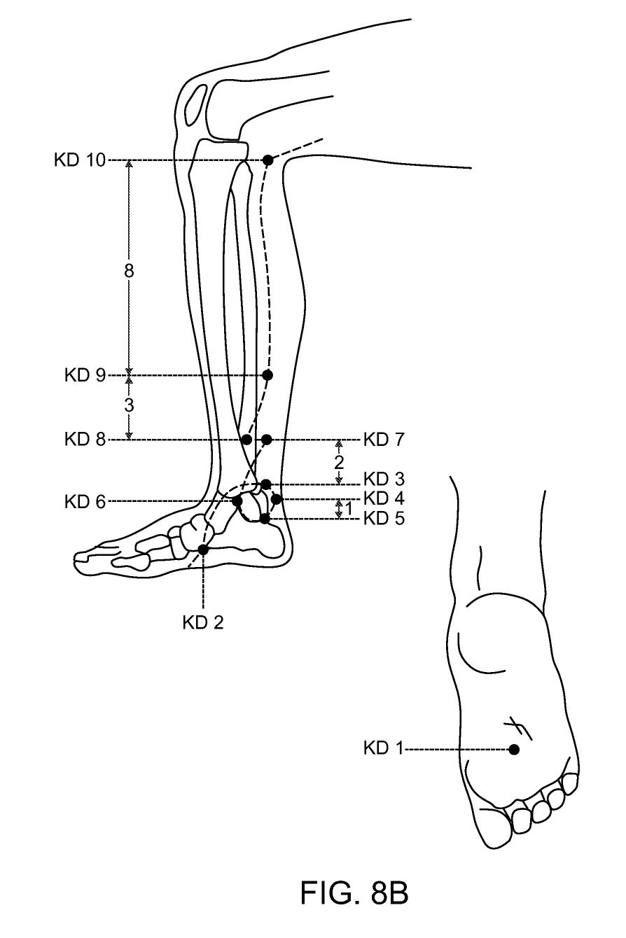

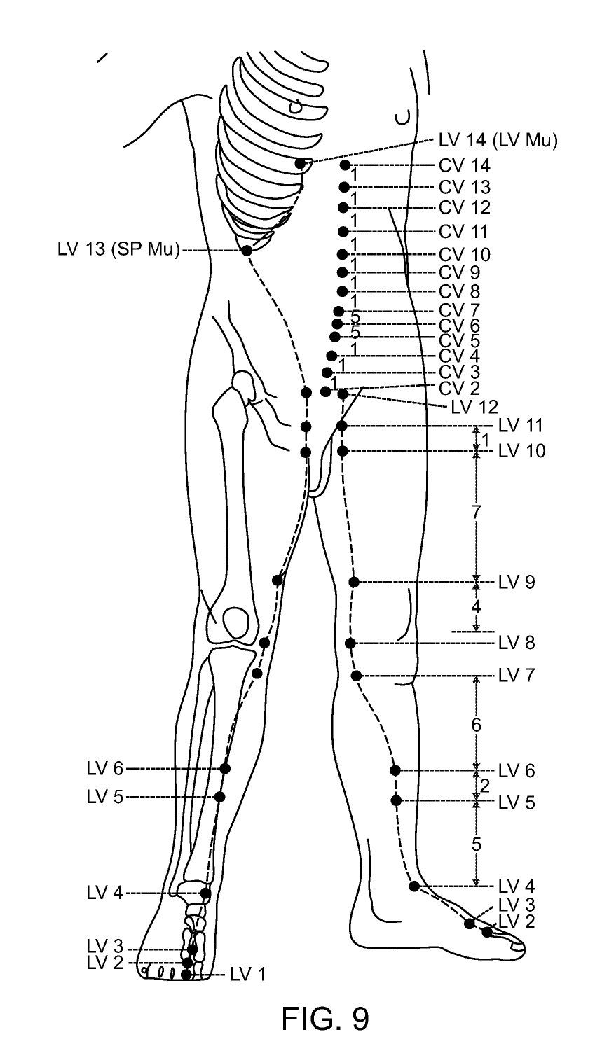

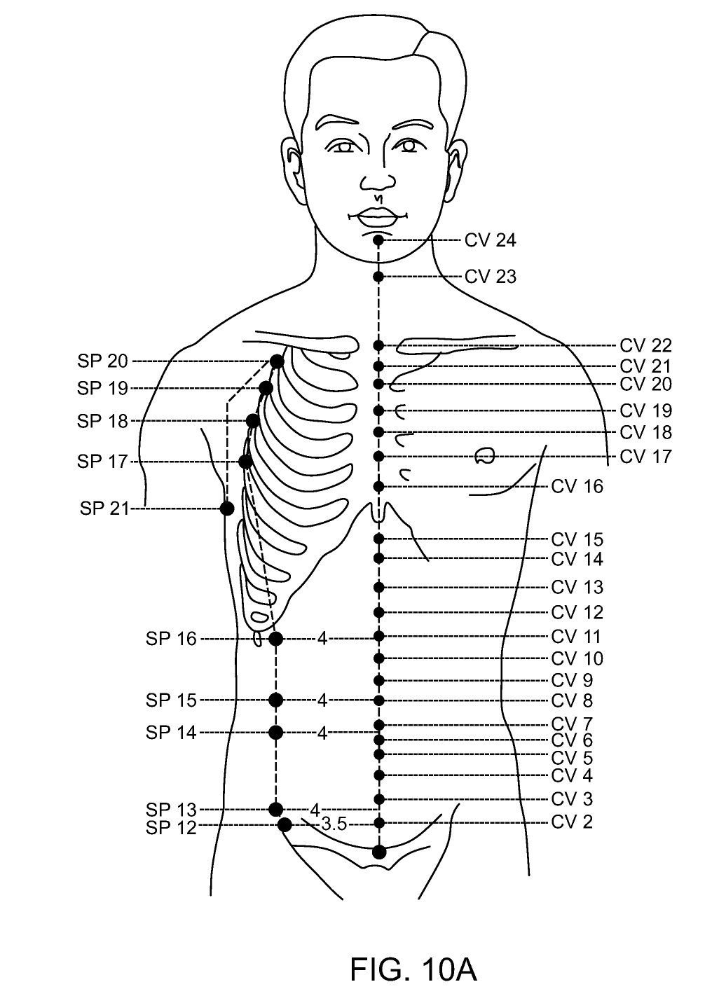

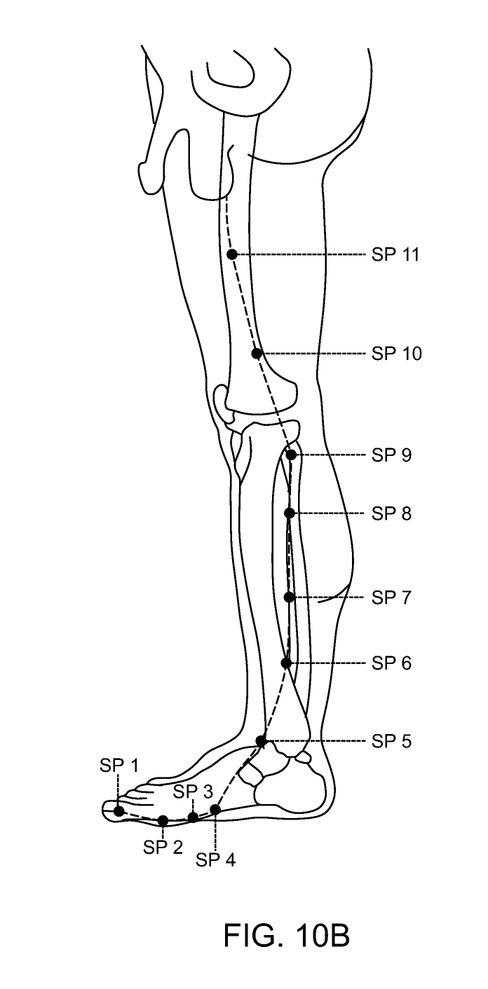

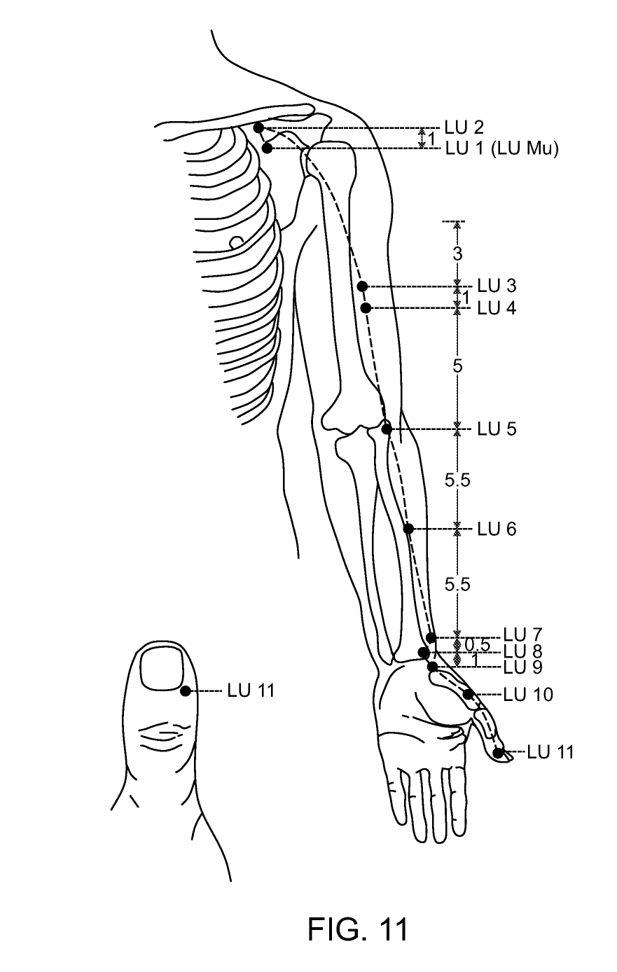

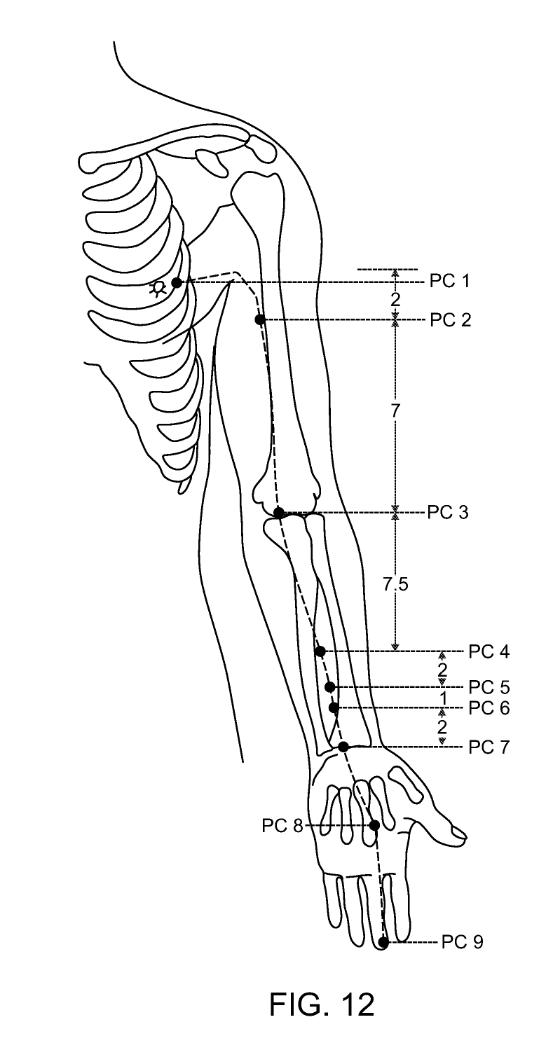

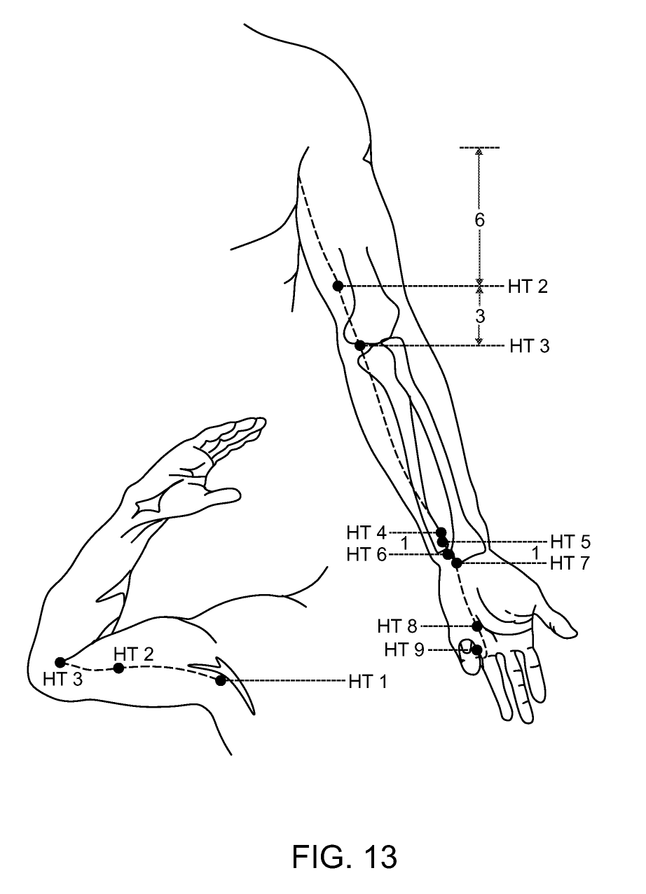

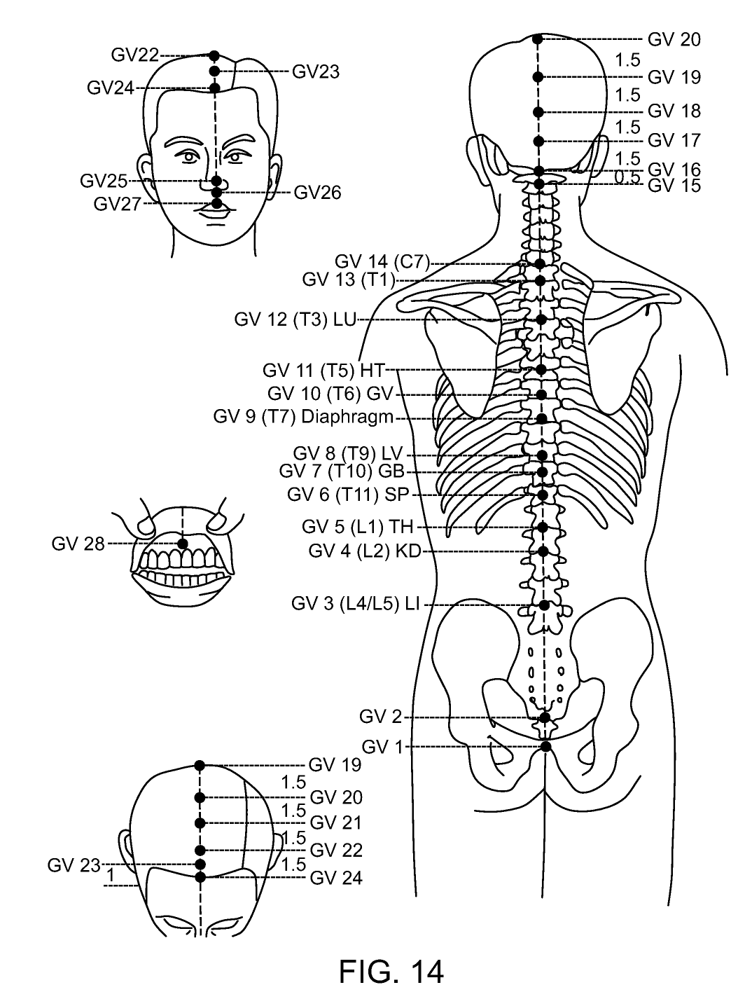

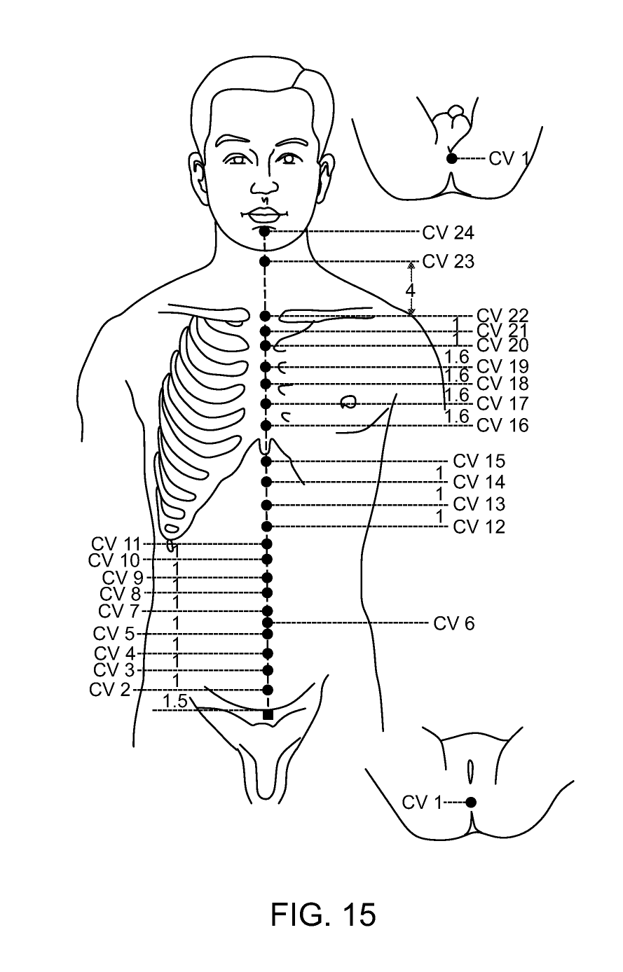

Each meridian includes numerous acupuncture points (about 400 acupuncture points in total), which are locations along the meridians in which the chi circulating through each channel rises close to the surface of the body (FIG. 1). FIGS. 2-13 are drawings showing acupuncture points along each of the twelve main meridians. FIGS. 14-15 are drawings showing acupuncture points along certain extraordinary meridians, the governing meridian and the conception meridian.

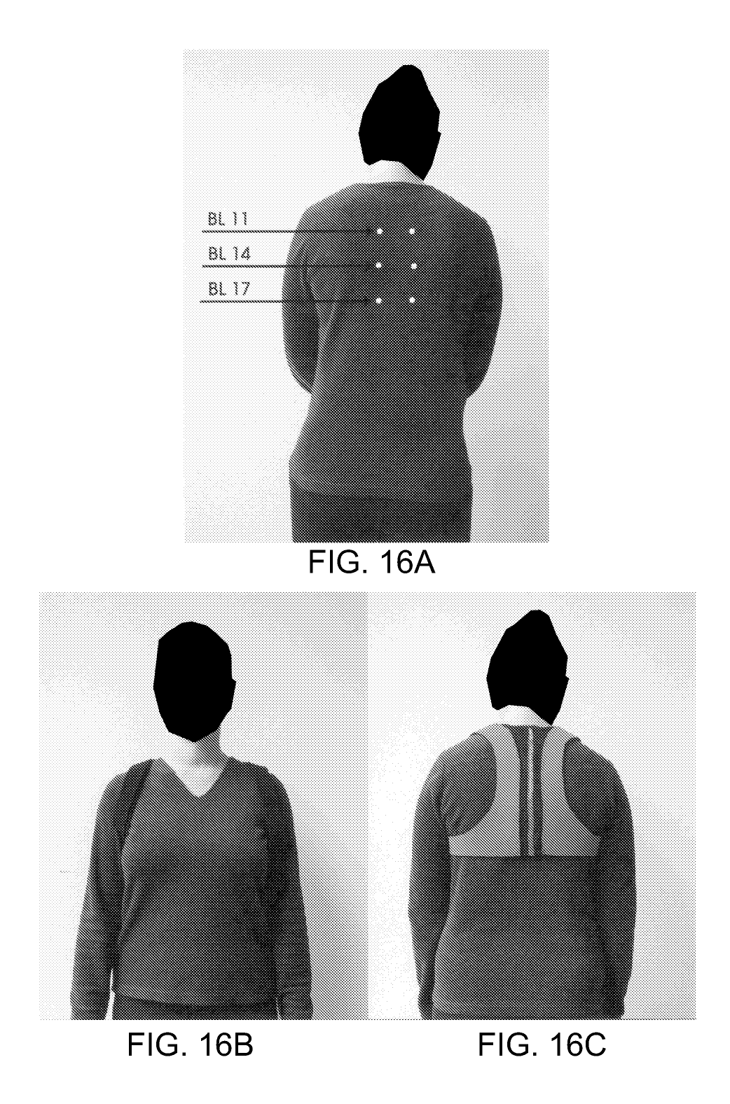

Stimulating acupuncture points across a plurality of meridians, activates a response, e.g., a healing response, in a body part of a person. In certain embodiments, the magnets are positioned circumferentially around the body part to result in periosteal stimulation of the body part. Periosteal stimulation stimulates multiple acupuncture points across multiple meridians and triggers a healing response in the body and treats the body part. In other embodiments, a particular set of acupuncture points are stimulated to trigger the healing response. For example, FIG. 16 panel A shows multiple acupuncture points along the bladder principle meridian (BL-11, BL-14, and BL-17) on the left and right sides of an upper back of a patient that will be stimulated by an upper thoracic brace in order to provide treatment to the muscles of the upper thoracic cavity of a patient.

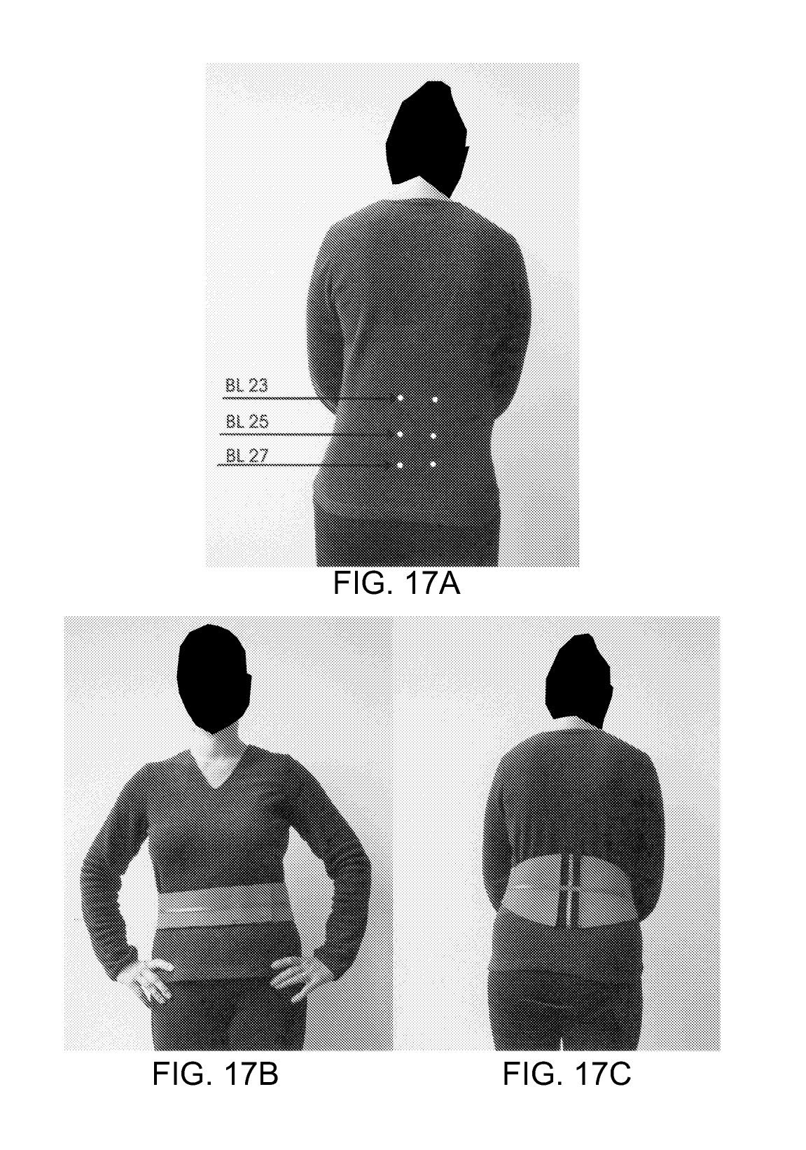

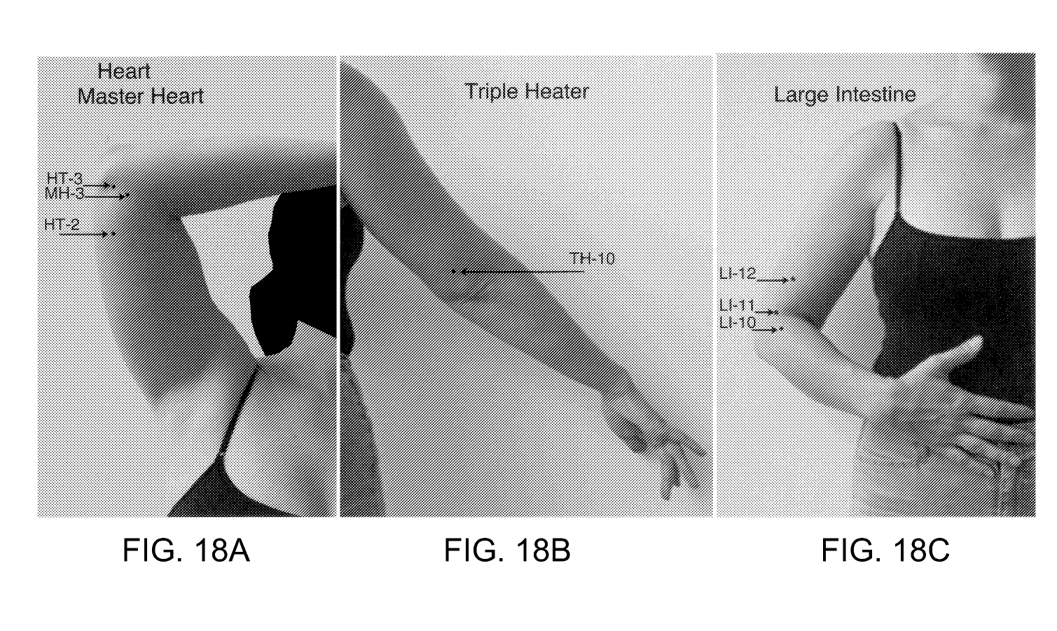

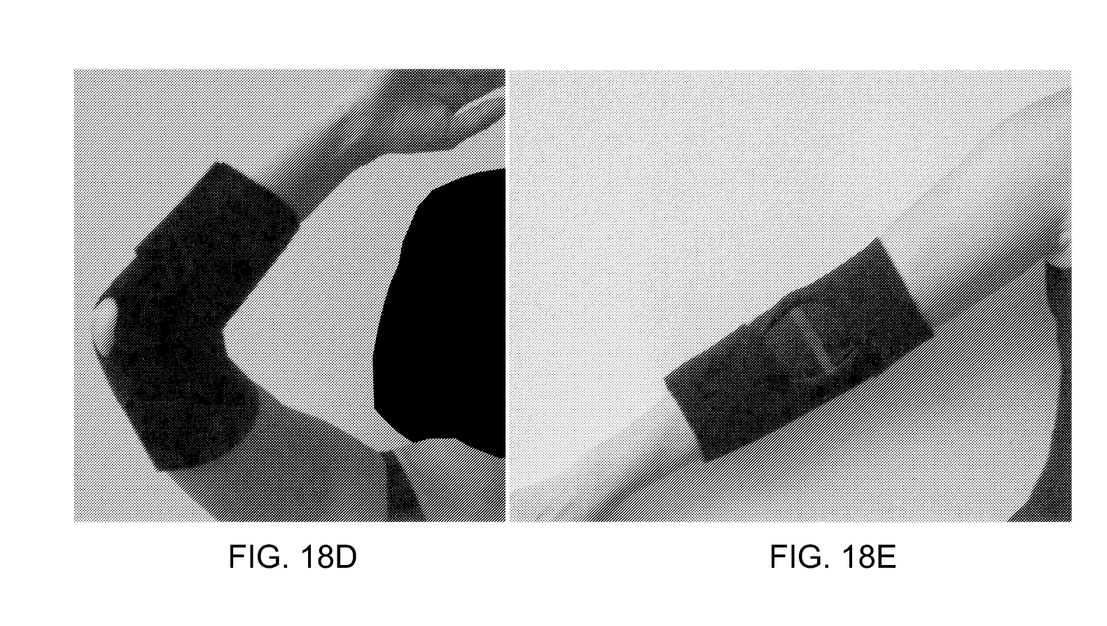

FIG. 17 panel A shows multiple acupuncture points along the bladder principle meridian (BL-23, BL-25, and BL-27) on the left and right sides of a lower back of patient that will be stimulated by a lumbar brace in order to provide treatment to the muscles of the lumbar of a patient. FIG. 18 panels A-C show multiple acupuncture points along the Heart meridian (HT-3 and HT-2), Master Heart meridian (MH-3), Triple Heater meridian (TH-10) and Large Intestine meridian (LI-12, LI-11, and LI-10) on an elbow of a patient that will be stimulated by an elbow brace in order to provide treatment to the elbow of a patient. In particular, this brace may be used to treat golf elbow or tennis elbow.

The acupuncture points along the Kidney meridian (KD-3) and the Bladder meridian (BL-60) on an ankle of a patient will be stimulated by an ankle brace in order to provide treatment to the ankle of a patient. Stimulation by this brace may also treat leg pain and heel pain, such as achilles tendonitis. The acupuncture point along the Stomach meridian (ST-36) and the Gall Bladder meridian (GB-25) on a knee of a patient will be stimulated by a knee brace in order to provide treatment to the knee of a patient.

The acupuncture points along the Bladder meridian (BL-23) and the Gall Bladder meridian (GB-25) around the L3 and L4 vertebra in the spine will be stimulated by a wrap in order to provide increased energy to a patient resulting in increased health and a sense of increased well-being.

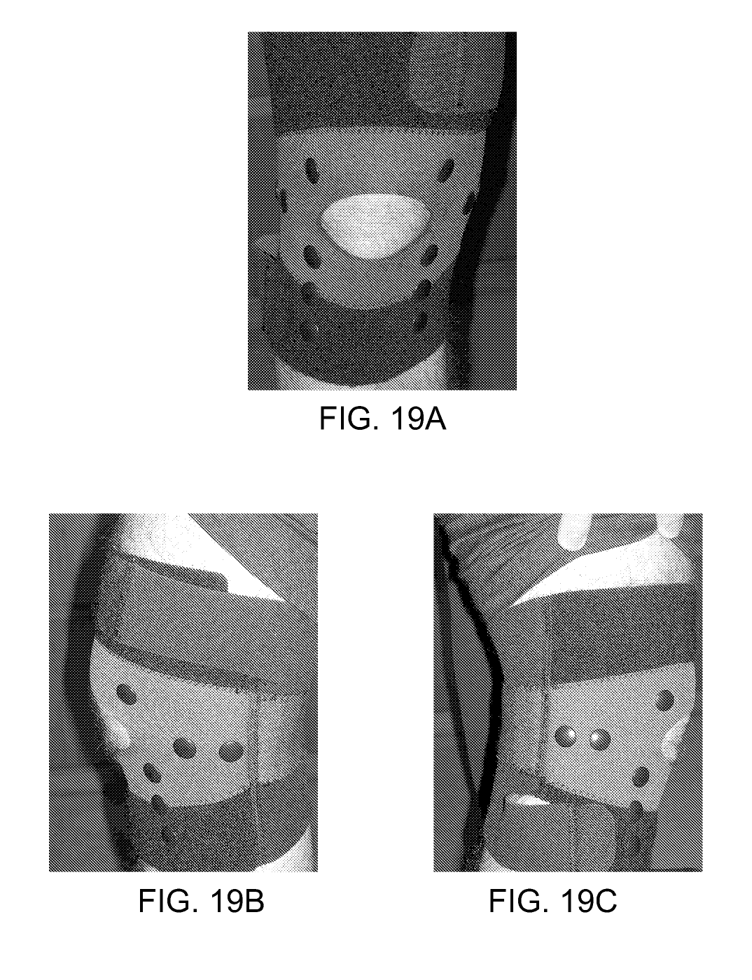

To ensure proper alignment of the devices of the invention, each of the devices includes an auto-alignment feature. For example, the knee brace includes a cut-out for the patella (FIG. 19, panel A). By aligning the cut-out in the brace with the patella, proper alignment of the magnets with the proper acupuncture points for stimulating a treatment response is ensured. The upper thoracic brace includes a vertical alignment member, e.g., a strip of ribbon, that runs the vertical length of the brace. The vertical alignment member is positioned to align with the spine of the back (FIG. 16, panel C). The lumbar brace includes both a horizontal and vertical alignment member. The horizontal alignment member lines-up with a waist of a patient at the top of the illiac crest (FIG. 17, panel B). The vertical alignment member lines-up with the spine (FIG. 17, panel C). The elbow brace includes a horizontal alignment member and a cut-out. The cut-out aligns with the outer elbow (FIG. 18, panel D), and the horizontal alignment member lines-up with an inside arm crease on the inside of the elbow (FIG. 18, panel E).

Another aspect of the invention provides devices that treat body parts by using magnets configured to result in periosteal stimulation of the body part. These devices include a carrier and a plurality of magnets, in which the magnets are positioned circumferentially around a body part to result in periosteal stimulation of the body part upon application of the device to the body part. This approach differs from traditional medical acupuncture by adapting the Craig Technique which uses electrical stimulation of acupuncture needles for the activation of points across meridians. Instead of contacting acupuncture points across meridians, the magnets are positioned circumferentially around the body part to result in periosteal stimulation of non acupuncture points to treat the body part, for example, for pain control or reduction of swelling (edema).

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

### EXAMPLES

### Example 1

### Treatment of Knee Pain

A 71 year old male presented for evaluation complaining of disabling right knee pain, resulting from known severe degenerative joint disease. The patient was fitted for a knee brace having magnets configured about the brace to surround the joint and to stimulate acupuncture points across multiple meridians. The magnets were arranged circumferentially about the knee to provide stimulation to multiple acupuncture points across the stomach and gall bladder meridians. FIG. 19 panels A-C show the subject wearing the knee brace, and the specific configuration of the magnets about the brace.

The knee brace was applied to the patient's knee by aligning the cut-out over the patella, ensuring alignment of the magnets to proper acupuncture points across the meridians. VELCRO closures were secured to hold the device in place. Treatment using the magnetic device involved a one hour treatment two or three times a week.

The patient reported rapid pain relief, which pain relief lasted for a significant period of time after treatment. The patient reported that the pain decreasing from an 8 out of 10 (pain scale with 10 being greatest pain) to a 2 out of 10 over the period of an hour while wearing the brace. The duration of pain relief lasted up to two days. Cumulative results with more sustained relief was noted after sequential treatments and this led to more effective stretching and a strengthening exercising program which prevents episodic exacerbations of symptoms.

### Example 2

### Treatment of Knee Pain

A 54 year old female presented for evaluation complaining of right knee pain resulting from patella fracture after a slip and fall. She was initially treated surgically, with rods and wire stabilization, which were subsequently removed. Upon removal, the patient reported increased pain in the right knee, and was forced to walk with the assistance of crutches.

Initial treatments involved four to five months of home based physical therapy. Furthermore, during this time, the patient continued with an ongoing light weight independent exercise program and began acupuncture treatments for pain as well. Despite undergoing rehabilitation, however, she noted persistent chronic and disabling right knee symptoms.

The patient was fitted for a knee brace having magnets configured about the brace to surround the joint and to stimulate acupuncture points across multiple meridians. The magnets were arranged circumferentially about the knee to provide stimulation to multiple acupuncture points across the stomach and gall bladder meridians.

The knee brace was applied to the patient's knee by aligning the cut-out over the patella, ensuring alignment of the magnets to proper acupuncture points across the meridians. VELCRO closures were secured to hold the device in place. Treatment using the magnetic device involved a one hour treatment two or three times a week.

The patient noted immediate improvement in knee swelling and pain which resulted in improvement in knee range of motion. As a result of improvement in mobility the patient was able to ambulate with a more normal gait and discontinued the assistive device for walking.

### Example 3

### Treatment of Muscles of the Upper Thoracic of a Patient

An upper thoracic brace is applied to an upper back of a patient to treat upper back pain (FIG. 16). The brace includes shoulder straps so the patient can wear the brace, and the brace also includes a carrier portion that holds the magnets. The magnets are arranged in the carrier to align with acupuncture points along the bladder principle meridian (BL-11, BL-14, and BL-17) on the left and right sides of an upper back (FIG. 16, panel A). The brace includes a vertical alignment member, in which proper alignment of the brace on the subject is achieved when the vertical alignment member of the brace lines-up with the spine (FIG. 16, panels B and C). The patient wears the brace, and the brace stimulates the acupuncture points to provide treatment to the muscles of the upper thoracic cavity of a patient.

### Example 4

### Treatment of Muscles of the Lumbar Muscles of a Patient

A lumbar brace is applied to a lower back of a patient to treat lower back pain (FIG. 17). The brace wraps around the patient and is secured to the patient by VELCO fasteners. The brace also includes a carrier portion that holds the magnets. The magnets are arranged in the carrier to align with acupuncture points along the bladder principle meridian (BL-23, BL-25, and BL-27) on the left and right sides of a lower back of the patient (FIG. 17, panel A). The brace includes both a horizontal alignment member and a vertical alignment member. Proper alignment of the brace on the patient is achieved when the horizontal alignment member lines-up with the waist of the patient at the top of the illiac crest, and the vertical alignment member lines-up with the spine (FIG. 17, panels B and C). The patient wears the brace, and the brace stimulates the acupuncture points to provide treatment to the lumbar muscles of the patient.

### Example 5

### Treatment of the Elbow

An elbow brace is applied to an elbow of a patient to treat elbow pain. The elbow brace includes VELCRO to fasten the brace around the elbow of the patient, and the brace also includes a carrier portion that holds the magnets. The magnets are arranged in the carrier to align with acupuncture points along the Heart meridian (HT-3 and HT-2), Master Heart meridian (MH-3), Triple Heater meridian (TH-10) and Large Intestine meridian (LI-12, LI-11, and LI-10) on the elbow of the patient (FIG. 18, panels A-C). The brace includes two alignment members, a cut-out and a horizontal alignment member. Proper alignment of the brace on the patient is achieved when the cut-out is fit over the outside of the elbow and the horizontal alignment member lines-up with the arm crease on the inside of the elbow (FIG. 18, panels D-E). The patient wears the brace, and the brace stimulates the acupuncture points to provide treatment to the elbow.

What is claimed is:

1. A device for treating a body part, the device comprising: a carrier and a plurality of magnets, wherein the magnets are configured to correspond with a plurality of acupuncture meridian points upon application of the device to the body part.

2. The device according to claim 1, wherein the magnets are positioned circumferentially around the body part.

3. The device according to claim 1, wherein at least one magnet is a rare earth magnet.

4. The device according to claim 1, wherein the magnets are magnetically oriented in the same direction about the body part.

5. The device according to claim 1, wherein the magnets are oriented in different magnetic directions about the body part.

6. The device according to claim 1, wherein one or more magnets contact the subject's skin.

7. The device according to claim 6, wherein at least a portion a magnet is covered with an inert metal.

8. The device according to claim 1, wherein the magnet comprises a planar surface, and the planar surface of the magnet is applied to the body part.

9. The device according to claim 1, wherein the magnet comprises a pointed surface, and the pointed surface of the magnet is applied to the body part.

10. The device according to claim 1, wherein the carrier member comprises an elastic material.

11. The device according to claim 1, wherein the carrier member is selected from the group consisting of a bandage, a wrap, and a patch.

12. The method according to claim 1, wherein the body part is an injured body part.

13. The method according to claim 1, wherein the body part is soft tissue.

14. A method for treating a body part of a subject, the method comprising:
applying a plurality of magnets to a body part of a subject, wherein the magnets are configured to correspond to a plurality of meridians, thereby to stimulate multiple points across the body part and trigger a response.

15. The method according to claim 14, wherein the magnets are positioned circumferentially around the body part.

16. The method according to claim 14, wherein at least one of the magnets is a rare earth magnet.

17. The method according to claim 14, wherein one or more magnets directly contact the subject's skin.

18. The method according to claim 17, wherein at least a surface of the magnet that contacts the skin is covered with an inert metal.

19. The method according to claim 14, wherein the magnet comprises a planar surface, and the planar surface of the magnet is applied to the body part.

20. The method according to claim 14, wherein the magnet comprises a pointed surface, and the pointed surface of the magnet is applied to the body part.

21. The method according to claim 14, wherein the magnets are oriented in the same direction about the body part.

22. The method according to claim 14, wherein the magnets are oriented in different directions about the body part.

23. The method according to claim 14, further comprising performing acupuncture on the body part.

24. The method according to claim 14, further comprising performing acupuncture or acupressure on an area of the subject that is different from the body part, wherein the area is associated with the body part through one or more meridians.

25. The method according to claim 14, wherein the body part is an injured body part.

26. The method according to claim 14, wherein the body part is soft tissue.

27. A device for treating a body part, the device comprising: a carrier and a magnet, wherein the magnet is configured to correspond with a plurality of acupuncture meridian points upon application of the device to the body part.

28. A device for treating a body part, the device comprising: a carrier and a plurality of magnets, wherein the magnets are positioned circumferentially around a body part to result in periosteal stimulation of the body part upon application of the device to the body part.
